(19) United States
(12) Patent Application Publication
(10) Pub. No.: US 2023/0293853 A1
(43) Pub. Date: Sep. 21, 2023

(54) INSERTION APPROACH DEVICE AND METHOD FOR USING THE SAME

(71) Applicant: TRG Media and Innovations Corp, Burnsville, MN (US)

(72) Inventors: James B. Williams, JR., Dunlap, IL (US); Shane Allan Kretsinger, Grantsburg, WI (US); Jamey R. Williams, Farmington, MN (US)

(21) Appl. No.: 17/715,933

(22) Filed: Apr. 7, 2022

Related U.S. Application Data
(60) Provisional application No. 63/320,939, filed on Mar. 17, 2022.

(51) Int. Cl. A61M 25/02

(52) U.S. Cl. CPC A61M 25/02; A61M 2025/024; A61M 2205/00

(57) ABSTRACT

A device and method for concurrently clamping a target location on a patient and providing an access guide for a needle and/or insertion equipment to the target location, wherein the device has a clasp body configured to provide a gripping tension to the target location and includes one or more apertures and corresponding one or more pathways for guiding the needle and/or insertion equipment to the gripped target location.

### CROSS-REFERENCE TO RELATED APPLICATION(S)

This application claims priority to U.S. provisional application No. 63/320,939, filed on Mar. 17, 2022, the content of which is hereby incorporated by reference in its entirety.

### BACKGROUND

The present invention relates to a device for guiding insertion of equipment into target site on a patient, and more specifically to a device for gripping the target site and providing a pathway to the target site for insertion of equipment with ease into a blood vessel or soft tissue of a patient.

Many medical procedures require the employment of devices such as catheters and other equipment intended to be introduced into blood vessels or other soft tissues, for a medical procedure.

Equipment insertions for selected procedures are difficult to achieve due to the nature of the insertion site, the vessel itself, the conditions surrounding the vessel, and/or dimensions of the cavity in which the insertion access needs to occur.

Successful insertion of catheters and other equipment is heavily reliant on the ability to introduce the equipment into the vessel or tissue at an angle appropriate for the specific procedure and maintaining the stationary attitude of the target itself.

Frequently, the success of the procedure is entirely dependent on the skill of the practitioner in achieving the required insertion “freehand”. Challenges faced by a practitioner are exacerbated when the target must be accessed through a blood vessel that tends to display reflex action, rolling, contraction, or slipping.

Presently, devices for employing equipment into a blood vessel or soft tissue provide a single angle of attack for insertion of sharps, such as needles. These devices similarly are unable to stabilize the blood vessel or soft tissue itself before, during, or after insertion.

### SUMMARY

An aspect of the present disclosure relates to a device for guiding approach and insertion of a needle and/or insertion equipment. The device has a clasp body, opposing clamping surfaces providing a cavity therebetween for receiving and holding a target location on a patient therein and between the opposing clamping surfaces, one or more insertion apertures in a first surface of the clasp body, and an exit aperture in a second surface of the clasp body. One or more pathways through the clasp body connect each of the one or more insertion apertures to the single exit aperture.

The device has a plurality of insertion apertures and corresponding plurality of pathways where each pathway is provided at a pathway angle to connect the insertion aperture to the exit aperture or to a second exit aperture, and wherein each pathway has a different pathway angle.

The clasp body has a cartridge, and the one or more insertion apertures, one or more pathways and the exit aperture or exit apertures are positioned on or in the cartridge. In one or more embodiments, the cartridge is removeable from the clasp body.

A geometry of the cartridge comprises curvature for specific redirection of the insertion equipment.

In one or more embodiments, the device has one insertion aperture and one curved pathway connecting the insertion aperture to the exit aperture.

The opposing clamping surfaces are each provided with a clamping interface and the clamping interfaces are flat, convex, concave, soft, toothed or combinations thereof.

The opposing clamping surfaces are each provided with a removable clamping interface such that the device comprises interchangeable clamping interfaces.

The opposing clamping surfaces are biased with a retention spring. The retention spring is comprised of a metal, flexible polymer, or combination thereof.

Extension levers are removably coupled to the clasp body for moving the opposing clamping surfaces.

One or more supplemental attachment anchors are provided on the clasp body.

The device comprises one or more surfaces that are transparent, translucent, or has optical clarity.

The device is comprised of plastic, acrylic, polymer, resin, metals or metal alloys, glass or combinations thereof.

The device is comprised of soluble, bioresorbable material, or combinations thereof.

Another aspect of the present disclosure relates to a method of capturing a target location on a patient for insertion of a needle and/or passing insertion equipment into the target location. The capture of the target location and insertion of the equipment includes securing a guide body to the target location and capturing the target location within a cavity in the guide body and directing a needle into a guide aperture in the guide body and through the guide body to an exit aperture, where the exit aperture provides access to the cavity in the guide body while clamping the guide body to the target location.

Sliding the guide aperture away from the target location with the insertion equipment retained in the target location may clear a field of view of the needle or insertion equipment.

Clamping the guide body to the target location can also be done to restrict or temporarily stop blood flow at the target location with the guide body.

The patient is a human or an animal and the target location includes a blood vessel, soft tissue or a combination thereof.

Yet another aspect of the present disclosure relates to a device for concurrently clamping a target location on a patient and providing an access guide for a needle and/or insertion equipment to the target location, wherein the device has a clasp body configured to provide a gripping tension to the target location and includes one or more apertures and corresponding one or more pathways for guiding the needle and/or insertion equipment to the gripped target location.

### BRIEF DESCRIPTION OF THE DRAWINGS

FIG. 1 is a perspective of an insertion approach device according to one or more embodiments described herein.

FIGS. 2-3 are top and bottom views of the insertion approach device.

FIGS. 4-5 are front and rear views of the insertion approach device, respectively.

FIG. 6-7 are side view of the insertion approach device.

FIG. 8 is a side perspective view of the insertion approach device.

FIG. 9 is a rear perspective view of the insertion approach device.

FIG. 10 is a top view of an access cartridge of the insertion approach device.

FIG. 11 is a bottom view thereof.

FIGS. 12 and 13 are rear and front perspective views of the access cartridge for the device and showing interior components of the cartridge in dashed lines.

FIGS. 14-22 illustrate embodiments interchangeable gripping interfaces with gripping surfaces for use with devices according to one or more embodiments herein.

FIG. 23 is a bottom right perspective view of a second embodiment of the insertion approach device.

FIG. 24 is a front view of the second embodiment of the device.

FIG. 25-26 are bottom left and top right perspective view of the second embodiment of the device.

FIG. 27 is a rear view of the second embodiment of the device.

FIG. 28 is a top left perspective view of the second embodiment of the device.

FIG. 29 is a top view of another embodiment of an access cartridge for the device and showing the interior components of the access cartridge.

FIGS. 30-31 are opposing side views of the access cartridge of FIG. 29.

FIGS. 32-33 are opposing perspective views of the access cartridge of FIG. 29.

FIG. 34 is a front side view thereof.

FIG. 35 is a perspective of another embodiment of an access cartridge for the device and showing the interior components of the access cartridge.

FIG. 36 is a perspective view of another embodiment of an insertion approach device.

### DETAILED DESCRIPTION

An insertion approach device is described herein. The insertion approach device improves success rates in the insertion of equipment in a target site. The device may be used to grasp and isolate a selected target site for insertion of equipment and further support insertion of the equipment. The device may be used to insert a needle into a blood vessel or soft tissue in a human or animal for providing a catheter or other medical/insertion equipment into the blood vessel, for example.

The insertion approach device captures and may further restrain the target site on a patient in a selected position for insertion of a needle, catheter or other equipment. The device can be used to capture or restrain a blood vessel and/or surrounding soft tissues and hold the blood vessel and/or surrounding tissue in a selected position. Reliable penetration of the blood vessel with catheters and other equipment is increased as the device further provides an approach cartridge that provides one or more controlled angles for entry or insertion of the needle, catheter or other equipment.

The device can be removably secured to a target area on a patient externally and is thus a non-implantable medical device in one embodiment. The device may also be secured to and capture a target area inside a patient and thus be employed subcutaneously and/or internally. In embodiments where the device may be used subcutaneously or otherwise inside the body of the patient, the device may be a removable device or may be constructed of a soluble or bioabsorbable material and/or remain inside the body after use. The device allows a practitioner to grasp and access blood vessels and/or soft tissues in various locations for various medical procedures and increases the number of practitioners who can successfully access difficult target sites.

The insertion approach device is a clamping or gripping device that may be provided in various sizes and shapes for use with different target sites and patient anatomy. The device comprises an approach cartridge. The approach cartridge may be integral with, or removably coupled to the device. The approach cartridge is a guide feature providing one or more guide pathways or approach apertures. Each approach aperture is a channel or pathway providing a selected or favorable angle of approach to the target site.

In embodiments where the approach cartridge is integral with the device and is not removeable therefrom, the approach apertures may be integral with a clasp body.

In embodiments where the approach cartridge is removable, the approach cartridge may also be slidable along the equipment, such as a catheter inserted into the target site through the device. This allows the approach cartridge to be slid along a length of the equipment and thus selectively moved out of a work space or manually bifurcated and removed. This construction allows for easy removal of the clamping device from the workspace as well. As the work space may be constrained, easy removal of the device described herein from the workspace during the procedure at hand is beneficial.

The approach cartridge may be provided as both a single or a multi-port approach cartridge. The multi-port approach cartridge provides a plurality of different insertion angles to the device and can be configured such that all or some included approach angles exit through the same point on a lower portion of the approach cartridge. This configuration allows the user to abandon one approach angle for another approach angle during the insertion attempt without changing the selected hardware location, fit, or insertion point.

The approach cartridge may further be provided with approach apertures having a degree of curvature. The arcuate length on the respective pathway allows the inserted equipment to follow a controlled path in which the equipment may be inserted in one direction and be redirected by the geometry of the approach aperture to follow a preset curve, in some cases reversing direction entirely prior to actual insertion into the target.

The device further comprises an entrapment cavity that is configured to hold the target site therewithin with a positive clamping force. The target site is held within the entrapment cavity allowing for insertion of selected insertion and/or medical equipment, such as catheters and similar devices, through the approach aperture and into the target site.

The insertion approach device allows medical procedures of this nature to be performed without a need to “chase or re-locate” an elusive blood vessel that may otherwise roll or “watermelon seed,” thus avoiding the perfect insertion that is required in preforming medical procedures.

In one or more embodiments, the device may further comprise a clamping interface. The interface may be substantially straight, concave, convex, or have an irregular shape and/or texture. The clamping interface may include a rigid, semi-rigid, or flexible direct gripping surface and may further include serrations, teeth, or other surface features for gripping and lifting the target site to be held or retained within the entrapment cavity. The clamping interface may further include one or more features configured to provide full or partial blood flow restriction or stoppage in a target vessel.

The device comprises a clasp body that may provide or apply clamping pressure and/or capture pressure using a spring device, a scissor style clamping provision, elastic, rubber, latex component for providing gripping tension or clamping force, or other mechanical means such as screw devices, vacuum pressure, cords, cables, wires, or ratcheting features to provide the appropriate gripping tension and/or clamping force to the clasp body.

In embodiments where a spring device is used, the spring device may be comprised of a metal, flexible polymer or other material sufficient to bias clamping arms around a target location on a patient.

The clasp body may further be provided with one or more of a variety of gripping interface shapes and designs. The device may further include a “soft jaw” feature comprising removably interchangeable gripping interfaces that may be introduced onto the clasp body.

In one or more embodiments, the device may be constructed from a suitably flexible material sufficient to allow clamping tension to be provided by a flexible bridge area between clamping surfaces of the device. Thus, the device may be a monolithic or single component device with a flexible portion located within the device that provides tension.

The device may be constructed with overall dimensions such that the device is capable of being inserted laparoscopically or by similar means into a patient for use.

The device may further comprise a supplemental attachment anchor for allowing an additional mechanism for attaching, stabilizing, and/or adding more clamping or penetrating force to the device. The supplemental anchor may be used when attaching the device to a target site on a limb, or other location along with commonly available medical supplies and equipment such as elastic tourniquet material or other means.

The device according to one or more embodiments described herein may additionally or alternatively be secured to or otherwise affixed to a patient by mechanisms including adhesive tapes and/or sutures. The sutures may be used externally and/or internally with respect to a target site and/or patient such that the clamp body could be affixed to the patient or affixed within the patient.

The device may also comprise one or more extension levers, where these extension levers may be fixedly or removably secured to the device body. The extension levers operate to open the device and thus open the entrapment cavity for application to the target site.

The device may be constructed from one or more medical grade plastics, polymers and similar compositions, metals and alloys, resins, and glass or glass composite materials, or any combination thereof. Iterations of the device may include the feature of being manufactured of a dissolving or bioresorbable material or similar. The device may comprise one surfaces or structures that are transparent, translucent, have a degree of optical clarity, substantially optically clear, or combinations thereof so as to prevent visual obstruction of a patient when in use.

The device may further be utilized with ultrasound guidance technology. The device may be configured with dimensions and/or constructed from materials which allow the device to be used in conjunction with ultrasound equipment to aid in determining location placement of the device and/or optimum placement of the device for needle introduction and/or equipment insertion to the target site. The device may further be configured for use with ultrasound guidance technology before, during, and after needle insertion.

As used herein, equipment may refer to a needle, medical and/or insertion equipment including but not limited to equipment for use in various medical procedures.

As used herein, the term target site or target location may refer to a location on a human or animal patient for insertion of a needle or medical equipment. Non-limiting examples of the target site or target location include blood vessels—arteries or veins—and/or soft tissue. These locations may be those used for access for the placement or insertion of medical equipment for various procedures. For example, the device may be used for the insertion of a catheter into an artery for placement of stents or may be used to isolate a tendon or ligament in other procedures. As used herein, the term target then may also refer to the blood vessel or soft tissue or other body component to which the equipment is inserted into and/or intended to access.

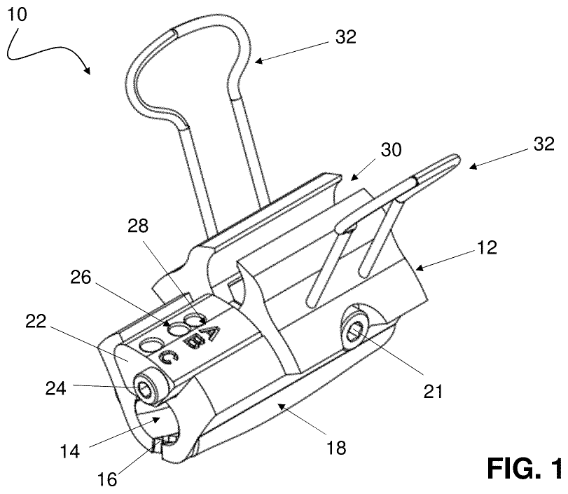

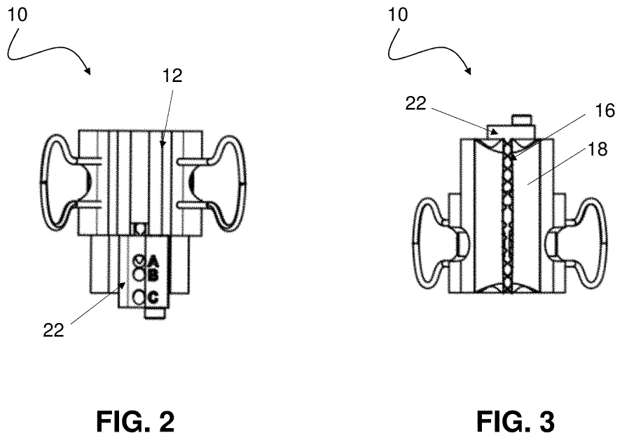

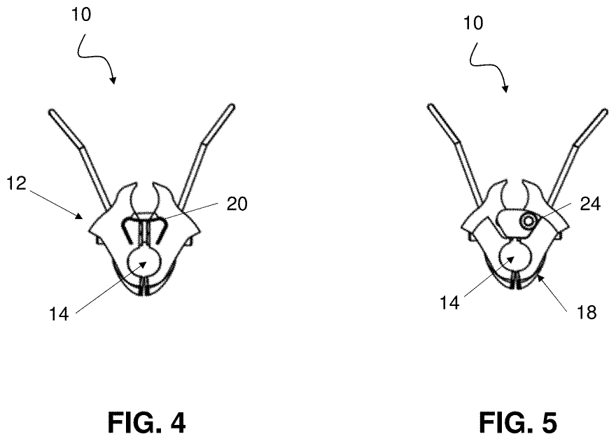

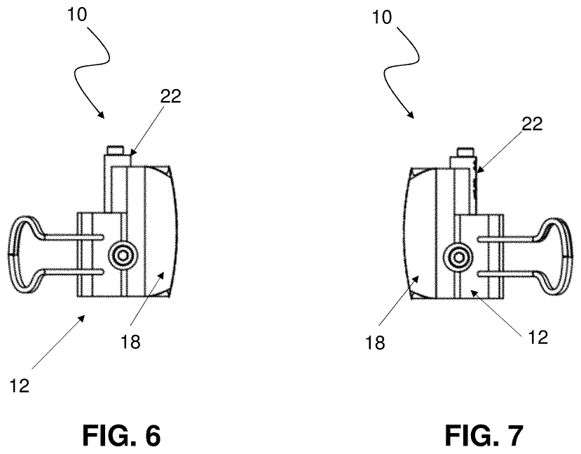

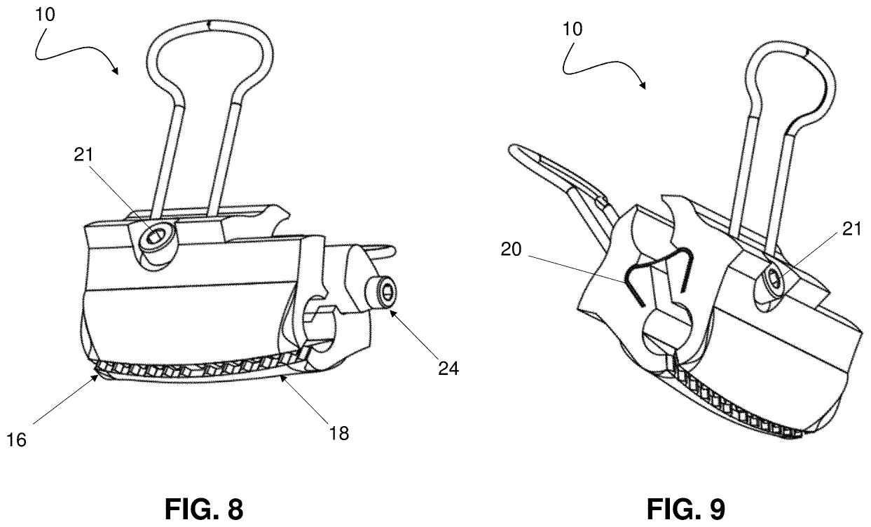

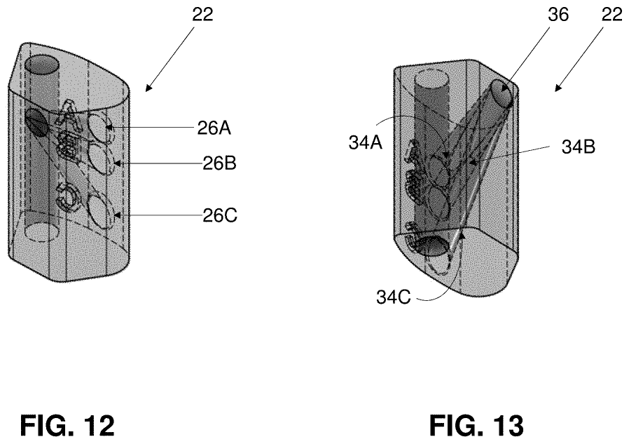

An embodiment of an insertion approach device 10 is illustrated in FIGS. 1-13. The device 10 comprises a clasp body 12, an entrapment cavity 14 and an approach cartridge 22. In brief, the clasp body 12 supports the entrapment cavity 14 which provides a space for receiving and holding a target site on a patient therein. Positioned above or at least partially over the entrapment cavity 14 is the approach cartridge 22 for providing access point(s) to the entrapment cavity 14 and the target site received therein.

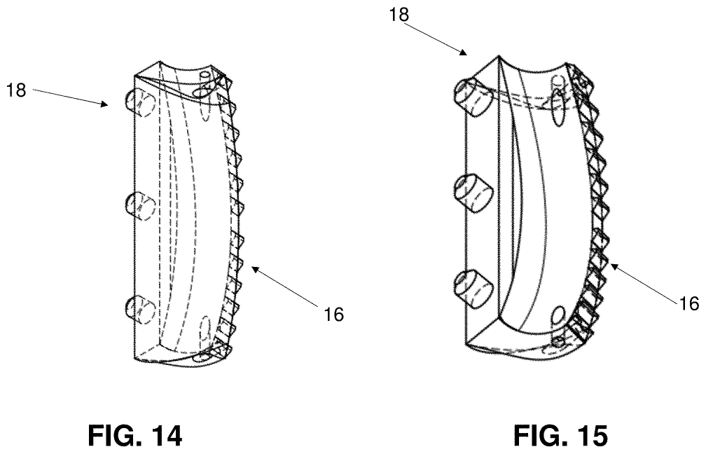

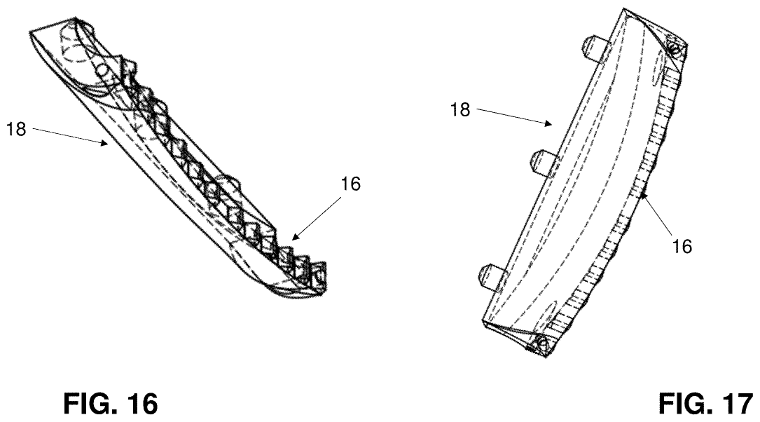

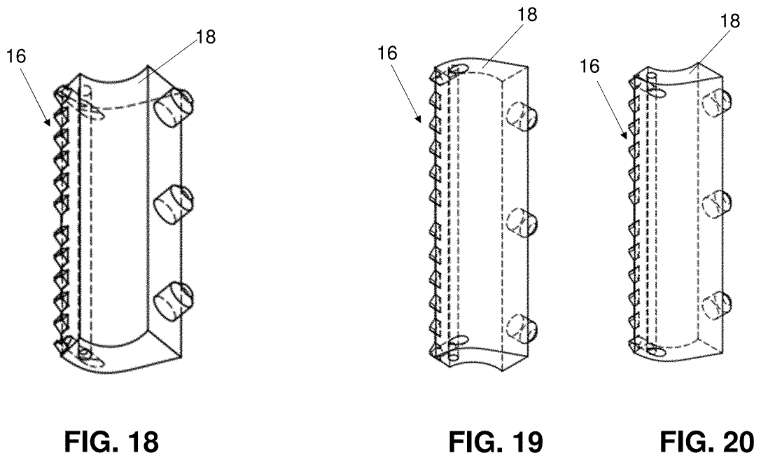

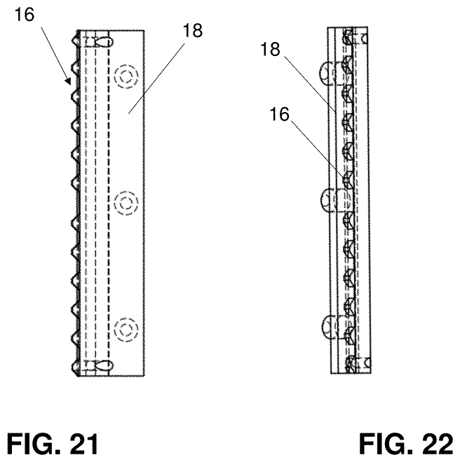

In further detail, the clasp body 12 operably supports a supplemental attachment anchor 30 and removable extension levers 32 operably secured on opposing sides of the clasp body 12. The levers 32 each have a handle portion that when forced together act to open the entrapment cavity 14 and increase the space between opposing gripping interfaces 18 of the clasp body 12. When the levers 32 are released the gripping interfaces 18 close together and grasp or otherwise secure a target site between direct gripping surfaces 16 positioned on outer faces of each of the opposing gripping interfaces 18. The direct gripping surface 16 may be removable from the gripping interfaces 18 of the clasp body 12 and interchangeable with different gripping surfaces 16. Non-limiting examples of different gripping interfaces 18 and direct gripping surfaces 16 for use the device 10 of one or more embodiments are illustrated in FIGS. 14-22.

In the embodiments illustrated, the opening and closing of the gripping interfaces 18 is biased by a retention device 20. In the illustrated embodiment, the retention device 20 is operably secured to the clasp body 12 by way of a fastener 38. The fastener 38 may extend through the clasp body 12 and capture or retain the retention device 20 therein.

The retention device 20 may be a spring retention device. As illustrated in the figures, the spring retention device 20 connects two opposing surfaces or walls of the gripping interface 18 to bias manual force applied to the levers 32 also connected to the opposing walls of the gripping interface 18 as illustrated in further detail, referring back to FIGS. 3 and 9.

The approach cartridge 22 is positioned and in some embodiments secured over the entrapment cavity 14 and between upper surfaces of the gripping interfaces 18 by way of an approach cartridge retention device 24. This device 24 may be a fastener for removably coupling the approach cartridge 22 to the clasp body 12. In the embodiment illustrated the fastener is a screw which couples the approach cartridge 22 to the clasp body 12.

The approach cartridge 22 in the embodiment illustrated comprises three approach apertures 26A, 26B, and 26C, where each aperture 26 may be provided with an identifier 28 to indicate an angle of approach through the respective aperture 26A, 26B, 26C to the entrapment cavity 14. In the embodiment of FIGS. 1-13, the angle of approach through each aperture 26A, 26B, 26C is different. The approach angles may range from about 0° to less than about 90° and may include pathways providing an insertion angle of the needle into the target in the range of about 5° to about 75° or in the range of about 20° to about 60°. Additional pathway geometry is contemplated and within the scope of this disclosure and includes direct and redirect pathways. While three apertures 26 are illustrated, it is contemplated and within the scope of this disclosure that the approach cartridge may be provided with a single aperture, two apertures, and three or more apertures.

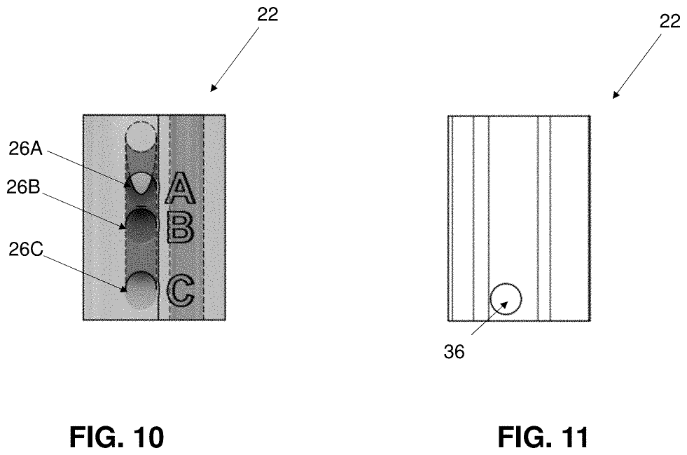

Referring next to FIG. 10-13, the approach cartridge 22 is illustrated in further detail. The approach apertures 26A, 26B, 26C and their respective angles of approach refer to the respective pathway 34A, 34B, 34C connecting each approach aperture 26A, 26B, 26C to a common or singular exit aperture 36. This exit aperture 36 opens to the entrapment cavity 14 and allows equipment inserted into a selected approach aperture 26A, 26B, 26C to pass through the exit aperture 36 and penetrate a target secured within the entrapment cavity 14. The exit aperture 36 leads to the entrapment cavity 14 and thus may be positioned on a patient facing or patient contact surface 42 of the device 10.

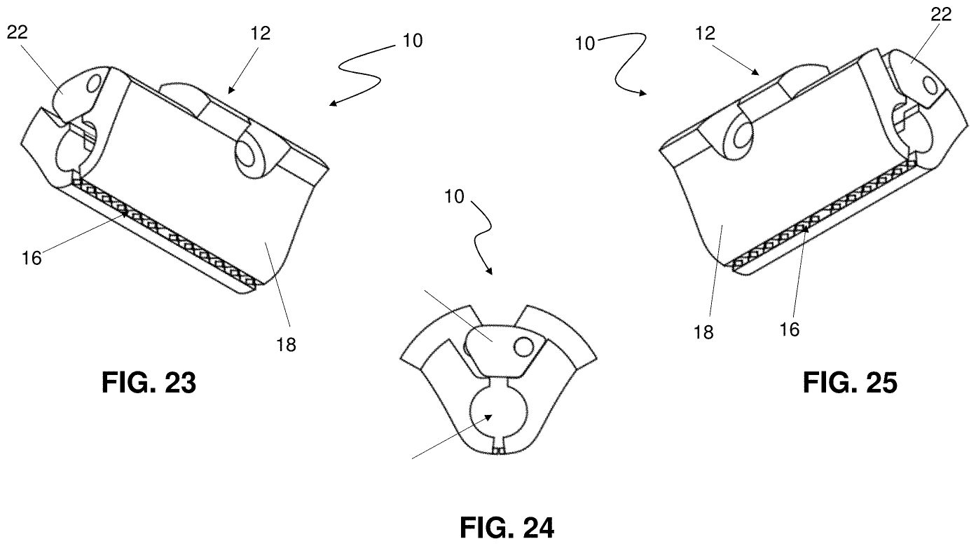

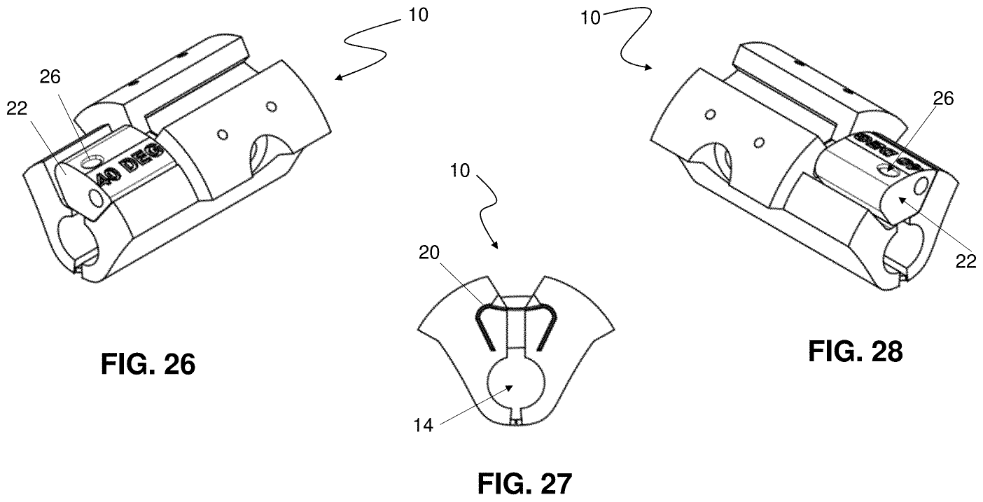

In an additional or alternative embodiment of the device 10 as illustrated in FIGS. 22-28, the device 10 is a single approach device having only one approach aperture 26 provided in the approach cartridge 22. The levers 32, for example, are removed from this device 10 for ease of illustration. The aperture 26 may be a straight approach aperture. A flat horizontal approach pathway at a substantially 0° angle with respect to the target site is also contemplated and within the scope of this disclosure and may be incorporated into a cartridge 22 having one or more approach apertures 26.

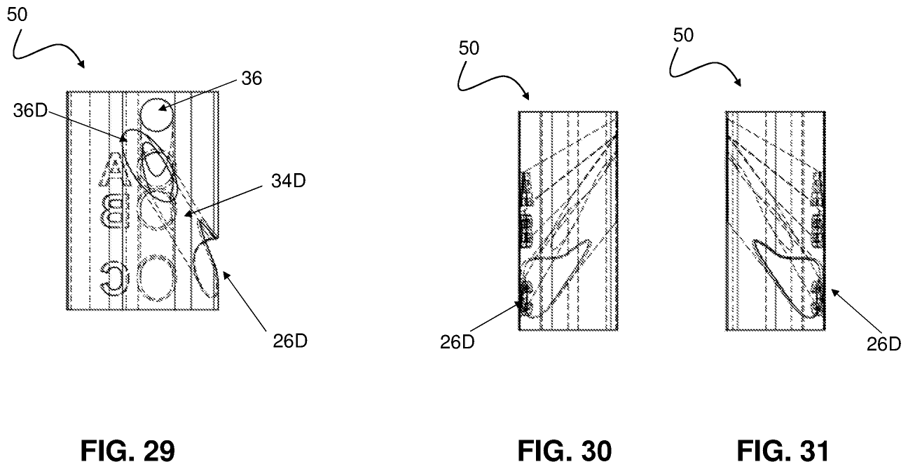

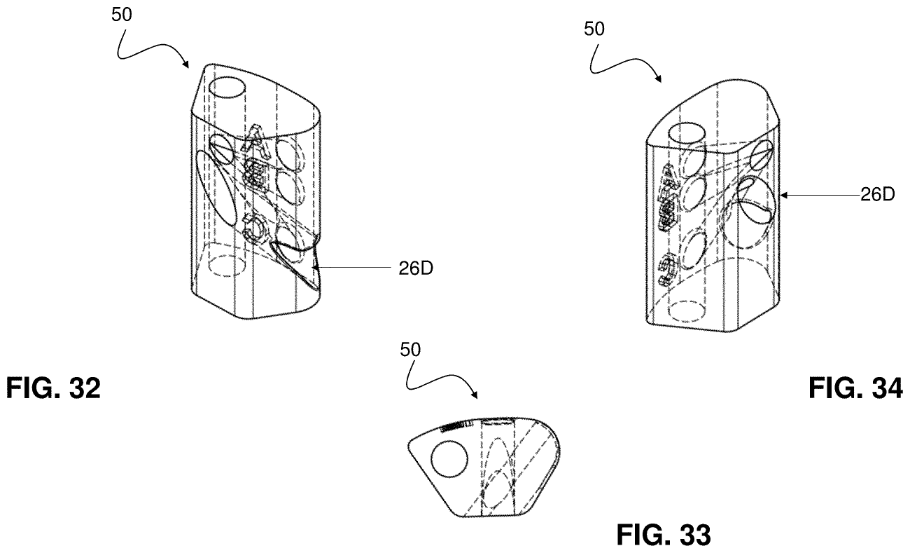

In an additional or alternative embodiment of the device 10, FIGS. 29-33 illustrate a lateral approach cartridge 50 isolated from the device 10. The approach cartridge 22 includes a lateral approach aperture 26D with lateral approach exit aperture 36D. The lateral approach aperture 26 may be provided on a surface of the approach cartridge 22 that is different from the surface which supports additional approach apertures 26A, 26B, or 26C. The lateral approach aperture 26D also includes a lateral pathway 34D that may terminate in the exit aperture 36 or in a second exit aperture 36D as illustrated.

In an additional or alternative embodiment of the device 10, FIG. 34 illustrates a directed approach cartridge 122 isolated from the device 10. The directed approach cartridge 122 provides a single directed approach aperture 126 with a curved pathway 134 to the exit aperture 136. In the directed approach cartridge 122, access to the directed approach aperture 126 may be from a front or side of the cartridge 122, such that the aperture 126 is provided in a vertical wall 110 of the cartridge 122. The exit aperture 136 is then provided on a bottom face of the cartridge 122 such that the directed approach aperture 126 and exit aperture 136 are provided along planes that may be substantially normal to one another and the apertures 126 and 136 are connected by a curved pathway 134 through the body of the cartridge 122. Thus, when in use, the approach aperture 126 may be substantially at or above the insertion location on the target site on the patient.

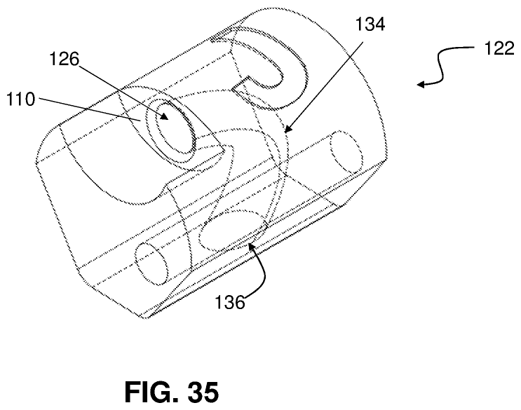

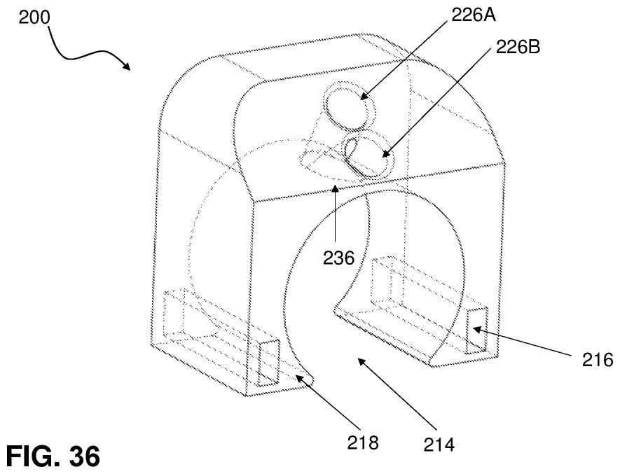

FIG. 35 illustrates an implantable or semi-implantable approach device 200 where this device may be provided inside or partially inside a patient's body. The device 200 comprises one or more approach apertures 226A, 226B having respective, different approach angles. The approach apertures 226A, 226B are connected by respective pathways having different approach angles to a single exit aperture 236 which opens directly into an entrapment cavity 214. This device 200 can be inserted directly over or around soft tissue or veins for direct access by selected equipment. The device 200 may further comprise one or more handling or anchor points 216 which provide apertures 216 in lower ends of sidewalls 218 of the entrapment cavity 214. These points 216 may be sutured or otherwise secured in place with the target or target site positioned in the entrapment cavity 214 and the equipment inserted and/or retained through the selected approach aperture 214 before and during a procedure. It is also contemplated that this device 200 may be constructed of a bioabsorbable or other biocompatible material such that the device 200 may be retained in the patient's body during and after use for insertion of equipment.

In one or more embodiments described herein, the device may also be a suture guide, for example for use in laparoscopic procedures. The device is configured to be secured in place, for example, clamped into place. Apertures in a base of the device, for example, the device illustrated in FIG. 35, can also be secured with the patient using one or more stitches. The apertures may be configured with a dimension and orientation such that stitches can be placed at right angles or even reverse angles. Once the stitches are secured, the device may be dissolved or manually bifurcated, and the sutures tightened in place in the body. This device and use thereof allows a practitioner to more appropriately and precisely places sutures in a patient than is presently possible.

An insertion approach device according to one or more embodiments described herein increases the ease of physically capturing, or otherwise restraining a target insertion site, examples of which include but are not limited to a blood vessel or soft tissue in a human or animal. The device according to one or more embodiments is a positively locating and guide device featuring one or more prescribed angle(s) for guiding equipment for insertion into a human or animal, at a position to allow improved insertion success.

Although the present disclosure has been described with reference to preferred embodiments, workers skilled in the art will recognize that changes may be made in form and detail without departing from the spirit and scope of the disclosure.

1. A device for guiding approach and insertion of a needle and/or insertion equipment, the device comprising:
a clasp body;
opposing clamping surfaces providing a cavity therebetween for receiving and holding a target location on a patient therein between the opposing clamping surfaces;
one or more insertion apertures in a first surface of the clasp body; and
an exit aperture in a second surface of the clasp body,
one or more pathways through the clasp body such that one pathway connects each of the one or more insertion apertures to the single exit aperture.

2. The device of claim 1, wherein the device comprises a plurality of insertion apertures and corresponding plurality of pathways where each pathway is provided at an angle to connect the insertion aperture to the exit aperture and wherein each pathway is provided at a different angle.

3. The device of claim 1 wherein the clasp body further comprises a cartridge and wherein the one or more insertion apertures, one or more pathways and the exit aperture are positioned on or in the cartridge.

4. The device of claim 3 wherein the cartridge is removeable from the clasp body.

5. The device of claim 3 wherein a geometry of the approach cartridge comprises curvature for specific redirection of the insertion equipment.

6. The device of claim 1 wherein the device has one insertion aperture and one curved pathway connecting the insertion aperture to the exit aperture.

7. The device of claim 1 wherein the opposing clamping surfaces are each provided with a clamping interface and the clamping interfaces are flat, convex, concave, soft, toothed or combinations thereof.

8. The device of claim 1 wherein the opposing clamping surfaces are each provided with a removable clamping interface such that the device comprises interchangeable clamping interfaces.

9. The device of claim 1 wherein the opposing clamping surfaces are biased with a retention spring.

10. The device of claim 1 wherein the retention spring is comprised of a metal, flexible polymer, or combination thereof.

11. The device of claim 1 and further comprising extension levers removably coupled to the clasp body for moving the opposing clamping surfaces.

12. The device of claim 1 and further comprising one or more supplemental attachment anchors.

13. The device of claim 1, wherein the device is transparent, translucent, or has optical clarity.

14. The device of claim 1, wherein the device is comprised of plastic, acrylic, polymer, resin, metals or metal alloys, glass or combinations thereof.

15. The device of claim 1, wherein the device is comprised of soluble, bioresorbable material, or combinations thereof.

16. A method of capturing a target location on a patient for insertion of a needle and/or passing insertion equipment into the target location, the method comprising:
securing a guide body to the target location and capturing the target location within a cavity in the guide body;
directing a needle into a guide aperture in the guide body and through the guide body to an exit aperture, where the exit aperture provides access to the cavity in the guide body while clamping the guide body to the target location.

17. The method of claim 16 and further comprising sliding the guide aperture away from the target location with the insertion equipment retained in the target location to clear a field of view of the insertion equipment.

18. The method of claim 16 and further comprising clamping the guide body to the target location and restricting or stopped blood flow at the target location with the guide body.

19. The method of claim 16 wherein the patient is a human or an animal and wherein the target location comprises a blood vessel, soft tissue or a combination thereof.

20. A device for concurrently clamping a target location on a patient and providing an access guide for a needle and/or insertion equipment to the target location, wherein the device has a clasp body configured to provide a gripping tension to the target location and includes one or more apertures and corresponding one or more pathways for guiding the needle and/or insertion equipment to the gripped target location.
